(19) United States
(12) Patent Application Publication
(10) Pub. No.: US 2025/0298012 A1
(43) Pub. Date: Sep. 25, 2025

(54) METHODS AND KITS FOR QUANTIFYING GLYCOSYLATED ANALYTES

(71) Applicant: Uniogen Oy, Turku (FI)

(72) Inventor: Jarkko Karvinen, Turku (FI)

(21) Appl. No.: 19/083,642

(22) Filed: Mar. 19, 2025

Related U.S. Application Data
(60) Provisional application No. 63/567,519, filed on Mar. 20, 2024.

(51) Int. Cl. G01N 33/543; G01N 33/574; G01N 33/68

(52) U.S. Cl. CPC G01N 33/54333; G01N 33/57488; G01N 33/6893; G01N 2333/42; G01N 2333/4724; G01N 2333/4725; G01N 2400/02

(57) ABSTRACT

A method for quantifying at least a first glycosylated analyte having a first glycan moiety and a first protein moiety, in a sample using an immunoassay, includes contacting the sample with a plurality of first magnetic particles, and a first antibody or an antigen-binding fragment thereof specific for said first protein moiety, labelled with a reactive enzyme; allowing the first magnetic particles to capture the first glycosylated analyte; allowing the first antibody or the antigen-binding fragment thereof to form a first immunocomplex with the captured first glycosylated analyte; separating captured analytes from any uncaptured analytes and unbound antibodies or antigen-binding fragments thereof; adding a first substrate and allowing it to react with the reactive enzyme of the labelled first antibody; measuring the intensity of the first detectable signal; and correlating the measured intensity of the first detectable signal to the quantity of the first glycosylated analyte in the sample.

### TECHNICAL FIELD

This disclosure relates to methods and kits for quantifying glycosylated analytes utilizing a magnetic particle-based immunoassay.

### BACKGROUND

Cancer remains one of the leading causes of death worldwide. Aberrant glycosylation is an established hallmark of cancer, often associated with cancer progression. Early detection and accurate diagnosis are crucial for effective cancer treatment and long-term patient survival. To meet this target, reliable, non-invasive methods for detecting and quantifying aberrantly glycosylated cancer antigens are needed.

### SUMMARY

The present disclosure relates to a method for quantifying in a sample one or more glycosylated analytes having a glycan moiety and a protein moiety as set forth in independent claim 1 and to a kit for use in the method as set forth in independent claim 24.

Further aspects, embodiments and details are set forth in the following figures, detailed description, examples, and dependent claims.

### BRIEF DESCRIPTION OF THE DRAWINGS

The accompanying drawings illustrate several embodiments of the disclosed subject matter, and together with the description, serve to explain principles of the disclosed compositions and methods.

FIG. 1A is a schematic illustration of a known method for analyzing glycosylated analytes. In the method, the analyte is captured using a specific antibody, such as an anti-CA125 antibody, immobilized on a solid surface, and traced with glycan-specific binders coated on a Eu+3-nanoparticle, which bind to glycan moieties of the target analyte, here to the glycan moieties of CA125. In the embodiment shown, the total duration of the method is 2 hours. TRF, time-resolved fluorescence.

FIG. 1B is an enlarged schematic illustration of the detectable immunocomplex formed in the method illustrated in FIG. 1A.

FIG. 2 is a schematic illustration of the principle of an embodiment of the present method having a single separation cycle. In the embodiment shown, the reactive enzyme is HRP, and the substrate is chemiluminescent.

FIG. 3A is a schematic illustration of the principle of another embodiment of the present method having two separation cycles. In the embodiment shown, the reactive enzyme is HRP, and the substrate is chemiluminescent.

FIG. 3B is an enlarged schematic illustration of the detectable immunocomplex formed in the methods illustrated in FIGS. 2 and 3A.

### DETAILED DESCRIPTION

A method for quantifying at least one glycosylated analyte in a sample using a magnetic particle-based immunoassay is provided. Each of the glycosylated analytes comprise a glycan moiety and a protein moiety. For each glycosylated analyte to be quantified, at least one of the glycan moieties and the protein moieties are different.

As used herein the terms “sample” and “test sample” are interchangeable and refer to a sample whose target analyte content is to be quantified. Suitable sample types are in particular samples of a bodily fluid, such as ascites fluid, seminal plasma, urine, blood such as plasma or serum, and peritoneal cavity fluid, obtained from a subject, preferably a human subject. A urine, blood, serum or plasma sample is the most preferred sample type to be used in the present method and all its embodiments.

It is to be understood that the number of similar glycan moieties in a glycosylated analyte may vary. However, for the sake of simple expression, said similar glycan moieties are referred to collectively as “a glycan moiety” regardless of their number in a single analyte. It is also to be understood that a glycosylated analyte may, in addition to said glycan moiety, contain additional glycans which are different from the glycan moiety to be employed in the present immunoassay.

In the present method, a glycosylated analyte to be quantified is captured via its glycan moiety through specific binding to a binder molecule coupled to a magnetic particle. The captured analyte is then detected by use of a labelled antibody or a labelled antigen binding fragment thereof, specific for the protein moiety of the analyte, the label being a reactive enzyme. When brought into contact with a compatible detectable substrate, the reactive enzyme acts on it creating a detectable signal.

As used herein, the term “antibody” refers generally to an immunoglobulin structure comprising two heavy chains and two light chains interconnected by disulfide bonds.

Antibodies can exist as intact immunoglobulins or as any of a number of well-characterized antigen-binding fragments or single chain variants thereof, all of which are herein encompassed by the term “antibody”. Non-limiting examples of said antigen-binding fragments include Fab fragments, Fab′ fragments, F(ab)2 fragments, F(ab′) 2 fragments, and Fv fragments. Said fragments and variants may be produced by recombinant DNA techniques, or by enzymatic or chemical separation of intact immunoglobulins as is well known in the art. Accordingly, the term “antigen-binding fragment” refers to any fragment of a monoclonal antibody regardless of its preparation technique, including enzyme used (e.g. pepsin, papain, bromelain), provided that the fragment has retained the antigen-binding specificity of the monoclonal antibody from which it derives.

Use of magnetic particles as capturers enables easy separation of the captured analytes from any uncaptured analytes or unbound antibodies or antigen-binding fragments thereof by applying an external magnetic field, for example, to “hold” the magnetic particles along with the captured analytes against a wall of a reaction container while a spend reaction fluid is removed therefrom. Optionally, the removal of the spend reaction fluid may involve one or more wash cycles. Once the external magnetic field is withdrawn, the magnetic particles are released. The number of the separation cycles, optionally combined with one or more wash cycles, may vary in the present method.

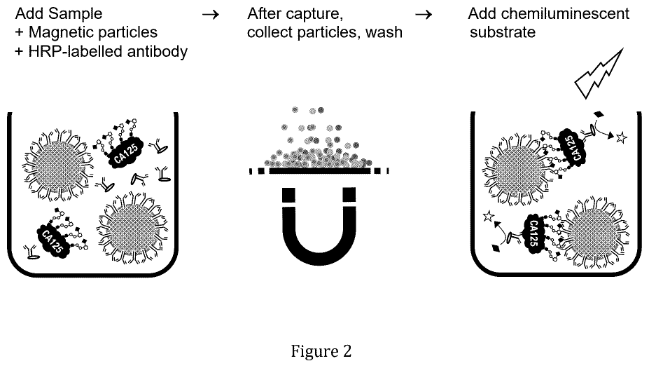

In an embodiment, the present method comprises one separation cycle, as illustrated in FIG. 2. In the embodiment, a sample whose content for at least one glycosylated analyte is to be quantified is contacted with a plurality of magnetic particles, each coupled with a plurality of same or different binder molecules specific for the glycan moiety of the analyte, and with an antibody or an antigen-binding fragment thereof specific for the protein moiety of the analyte, labelled with a reactive enzyme. The magnetic particles are then allowed to capture the glycosylated analyte through specific binding to the glycan moiety of the analyte, and the labelled antibody or an antigen binding fragment thereof is allowed to form an immunocomplex with the captured glycosylated analyte through specific binding to the protein moiety of the analyte. The immunocomplex is then separated from any uncaptured analytes and unbound antibodies or antigen-binding fragments thereof using an external magnetic field, optionally combined with one or more wash cycles. A substrate compatible with the reactive enzyme is then added and allowed to react with the reactive enzyme, thereby resulting in a detectable signal. The intensity of the detectable signal is then measured and correlated to the quantity of the glycosylated analyte in the sample.

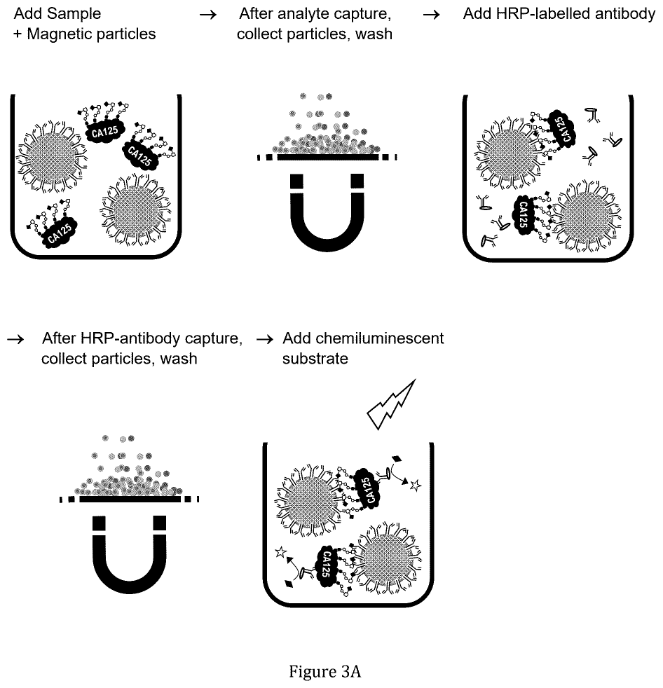

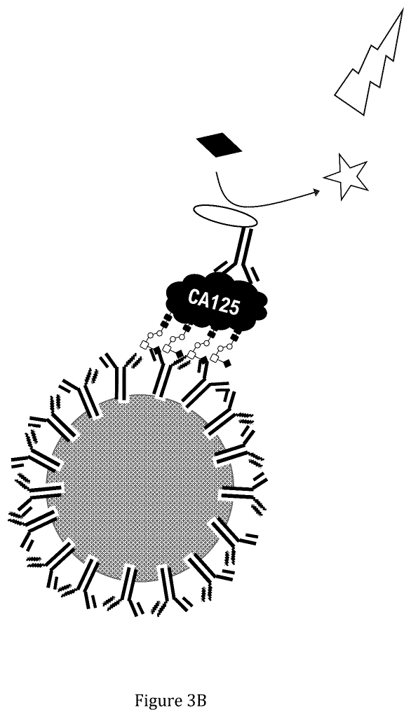

In an embodiment, the present method comprises two separation cycles, as illustrated in FIG. 3. In the embodiment, a sample whose content for at least one glycosylated analyte is to be quantified is contacted with a plurality of magnetic particles, each coupled with a plurality of same or different binder molecules specific for the glycan moiety of the analyte. The magnetic particles are then allowed to capture the glycosylated analyte through specific binding to the glycan moiety of the analyte. The captured analyte is then separated from any uncaptured analytes using an external magnetic field, optionally combined with one or more wash cycles. The captured analyte is then contacted with an antibody or an antigen-binding fragment thereof specific for the protein moiety of the analyte, labelled with a reactive enzyme, and allowed to form an immunocomplex with the captured analyte through specific binding to the protein moiety of the analyte. A substrate compatible with the reactive enzyme is then added and allowed to react with the reactive enzyme, thereby resulting in a detectable signal. The intensity of the detectable signal is then measured and correlated to the quantity of the glycosylated analyte in the sample.

Both of the above-mentioned embodiments may be multiplexed to detect more than one, such as two or three, different glycosylated analytes simultaneously. In such instances, at least one of the glycan moieties and the protein moieties of a glycosylated analyte are different from those of the other glycosylated analytes. In an embodiment, a first and a second analyte may have the same protein moieties but differ with respect to their glycan moieties. In another embodiment, a first and a second analyte may have the same glycan moieties but differ with respect to their protein moieties. In a further embodiment, a first and a second analyte may differ from each other with respect to their glycan and protein moieties. In still further embodiments involving also a third analyte, either the glycan moiety or the protein moiety, or both must be different from those of the first and the second analyte. In each case, different reactive enzymes and different substrates compatible with the reactive enzyme in question must be used for each glycosylated analyte to be quantified.

Any reactive enzymes available in the art, suitable for use in immunoassays may be employed in the present method. Non-limiting examples of suitable reactive enzymes include alkaline phosphatase (AP) or horseradish peroxidase (HRP).

Both chromogenic and chemiluminescent substrates may be used in the present method. When reacted with a compatible reactive enzyme, a chromogenic substrate converts into a colored substance enabling a colorimetric detection. A chemiluminescent substrate, in turn, results in a luminescent emission signal when reacted with a compatible reactive enzyme.

In an embodiment, the substrate is selected from 5-bromo 4-chloro-3-indolyl phosphate (BCIP), p-nitrophenyl phosphate (pNPP), derivatives of adamantyl 1,2-dioxetane phosphate, 3-(2′-spiroadamantane) 4-methoxy-1-4 (3″-phosphoryloxy)phenyl 1,1,2-dioxetane, 2,2′-azinobis (3 ethylbenzothiazoline-6-sulfonic acid) (ABTS) combined with hydrogen peroxide, 3,3′,5,5′-tetramethylbenzidine (TMB), diaminobenzidine (DAB), luminol, isoluminol, other luminol-based substrates lucigenin, pholasin and acridan-based substrates.

Suitable chromogenic substrates for AP include, but are not limited to, 5-bromo 4-chloro-3-indolyl phosphate (BCIP) and p-nitrophenyl phosphate (pNPP), whereas suitable chemiluminescent substrate for AP include, but are not limited to, derivatives of adamantyl 1,2-dioxetane phosphate, and 3-(2′-spiroadamantane) 4-methoxy-1-4 (3″-phosphoryloxy) phenyl 1,1,2-dioxetane. Further suitable chromogenic and chemiluminescent substrates for AP are readily available in the art.

Suitable chromogenic substrates for HRP as the reactive enzyme include, but are not limited to, 2,2′-azinobis (3 ethylbenzothiazoline-6-sulfonic acid) (ABTS) combined with hydrogen peroxide, 3,3′,5,5′-tetramethylbenzidine (TMB), diaminobenzidine (DAB), whereas suitable chemiluminescent substrates for HRP include, but are not limited to, luminol, isoluminol, lucigenin, pholasin and acridan-based substrates. Further suitable substrates for HRP are readily available in the art. Since hydrogen peroxide is important for the enzymatic activity of HRP, it is typically added into the reaction together with its substrate.

Techniques for labeling antibodies or antigen-binding fragments thereof with reactive enzymes are readily available in the art.

Also techniques and equipment for detecting and measuring intensity of signals generated when a reactive enzyme acts on its chromogenic or chemiluminescent substrate are readily available in the art. The measured signal intensity may then be correlated to the concentration of the glycosylated analyte in the sample. This may be accomplished by plotting a calibration curve based on known concentrations of the glycosylated analyte on the x-axis and corresponding signal intensities on the y-axis, and determining the quantity of the glycosylated analyte in the sample using the calibration curve and the measured signal intensity.

As explained above, the present method may include one or more optional wash cycles in order to remove any analytes present in the test sample that are not capable of specific binding to a binder molecule coupled to the surface of a magnetic particle and/or any antibodies not specifically bound to the protein moiety of a target analyte.

Appropriate wash solutions and conditions (e.g. time and temperature) are known to those skilled in the art.

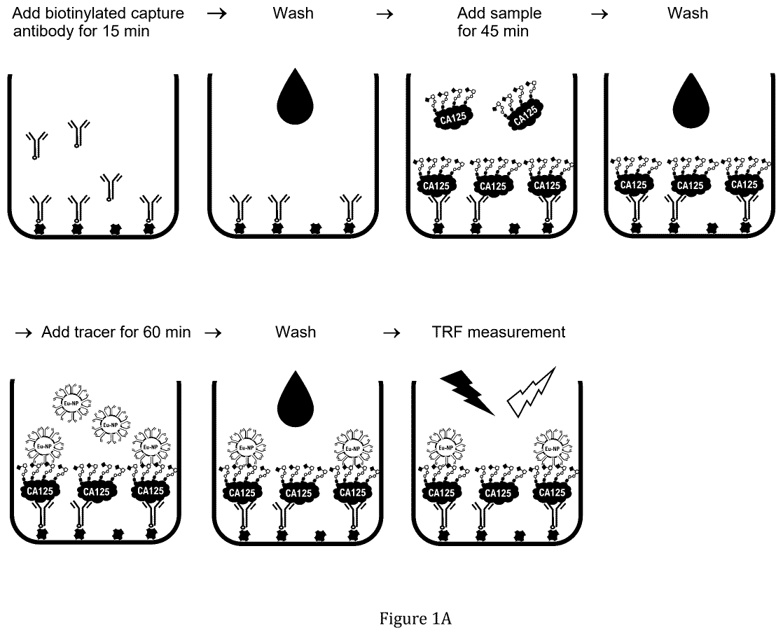

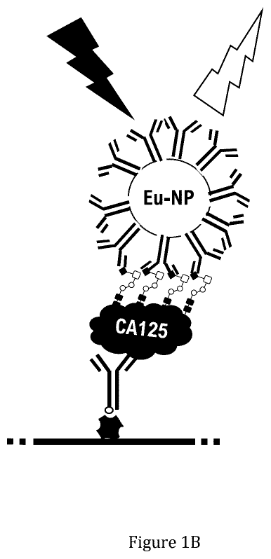

Except for the optional wash cycles, the present method may be carried out in one or more buffer solutions which are not particularly limited. Appropriate buffer solutions and incubation conditions (e.g. time and temperature) are either known or can be easily determined by those skilled in the art. In an embodiment, the present method may be carried out in about 60 minutes, preferably in about 40 minutes, and more preferably in about 20 minutes. These time frames are significantly shorter than the time required for carrying out the already known method illustrated in FIG. 1A, which takes about 2.5 hours.

Magnetic particles suitable for use in the present method may also be referred to as magnetic beads or magnetic microspheres. Magnetic particles commonly used in the art having a diameter ranging from about 0.1 μm to about 5 μm, preferably from about 0.3 μm to about 3 μm, may be used.

Preferably the magnetic particles to be used in the present method are made of materials that exhibit paramagnetic or superparamagnetic behavior. This means that the particles are not magnetic outside a magnetic field, but are magnetized quickly under the action of an external magnetic field. This enables the magnetic particles to remain in suspension when no magnetic field is applied to them and to collect the particles together quickly, along with anything they are bound to, with the help of an external magnetic field. Preferably, the magnetic particles have zero or only neglectable residual magnetism after the magnetic field is withdrawn, making it possible to easily get the magnetic particles back into a suspension.

Magnetic particles suitable for use in the present invention have a paramagnetic or superparamagnetic core and a non-magnetic, usually polymeric shell. Both paramagnetic and superparamagnetic materials rely on iron oxide-based materials, such as magnetite (Fe₃O₄). Without limitation, one purpose of the shell is to protect the core from reacting with hydrogen peroxide (if present) or other components in the immunoassay, thus preventing distortion in the detectable signal to be measured. Optionally, the shell may be coated with one or more functional groups carboxylic acid groups, hydroxyl groups, amino groups, thiol groups, and/or tosyl groups; with one or more proteins such as streptavidin, protein A, and/or protein G; and/or with biotin, to facilitate coupling of a plurality of same or different binder molecules on the surface the magnetic particle. Means and methods for said coupling are known to those skilled in the art, and is not particularly limited.

In an embodiment, the magnetic particles are composites having a magnetic core and a non-magnetic polymeric shell.

In a further embodiment, the polymeric shell is preferably polystyrene, optionally coated with one or more functional groups such as carboxylic acid groups, hydroxyl groups, amino groups, thiol groups, and/or tosyl groups; with one or more proteins such as streptavidin, protein A, and/or protein G; and/or with biotin.

Glycan-specific binder molecules suitable for use in the present method include, but are not limited to, lectins and various glycan-specific antibodies or antigen-binding fragments thereof. Magnetic particles for use in capturing a glycosylated analyte of interest may include a plurality of same or different binder molecules, provided that the different binder molecules are specific for the same glycan moiety of the target analyte of interest. Accordingly, a magnetic particle may thus contain, for example, either a plurality of lectins, a plurality of glycan-specific antibodies, or a mixture of both coupled on the surface of said particle.

In an embodiment, the method is based on a non-competitive immunoassay.

In an embodiment, the present method is to be carried out in a reaction container, such as in a microtiter plate or a reaction chamber of a microfluidistic device. It also envisaged that the method may be formulated to lateral flow format.

In an embodiment, the glycosylated target analyte is CA125-STn having a sialyl Tn antigen (STn) as the glycan moiety and cancer antigen 125 (CA125) as the protein moiety. In an embodiment for quantifying said CA125-STn, the binder molecule is an anti-STn antibody, macrophage galactose-type lectin (MGL), or both, and the antibody specific for the protein moiety is an anti-CA125 antibody or an antigen-binding fragment thereof.

In an embodiment, the glycosylated target analyte is CA125-Tn having a Tn antigen (STn) as the glycan moiety and CA125 as the protein moiety. In an embodiment for quantifying said CA125-Tn, the binder molecule is an anti-Tn antibody, MGL, or both, and the antibody specific for the protein moiety is an anti-CA125 antibody or an antigen-binding fragment thereof.

In an embodiment, the glycosylated target analyte is a CEA glycovariant having CEA as the protein moiety and a glycan which is capable of specific binding to mannose binding lectin (MBL), Dendritic Cell-Specific Intercellular adhesion molecule-3-Grabbing Non-integrin (DC-SIGN) or to MGL as the glycan moiety. In an embodiment for quantifying said CEA glycovariant, the binder molecule is MBL, DC-SIGN, MBL or an antibody capable to specific binding to the same glycan moiety as MBL, DC-SIGN or MBL; and the antibody specific for the protein moiety is an anti-CEA antibody or an antigen-binding fragment thereof.

In an embodiment, the glycosylated target analyte is a CA15-3 glycovariant having CA15-3 as the protein moiety and a glycan which is capable of specific binding to MGL or wheat germ agglutin (WGA) as the glycan moiety. In an embodiment for quantifying said CA15-3 glycovariant, the binder molecule is MGL, WGA, or an antibody capable to specific binding to the same glycan moiety as MGL or WGA; and the antibody specific for the protein moiety is an anti-CA15-3 antibody or an antigen-binding fragment thereof.

In an embodiment, the glycosylated target analyte is a prostate-specific antigen (PSA) glycovariant having PSA as the protein moiety and a glycan which is capable of specific binding to MGL as the glycan moiety. In an embodiment for quantifying said PSA glycovariant, the binder molecule is MGL, or an antibody capable to specific binding to the same glycan moiety as MGL; and the antibody specific for the protein moiety is an anti-PSA antibody or an antigen-binding fragment thereof.

In an embodiment, the glycosylated target analyte is an integrin glycovariant having integrin selected from integrin subunit alpha 3 (ITGA3), integrin subunit alpha 1 (ITGA1), integrin subunit alpha 2 (ITGA2), integrin subunit alpha 4 (ITGA4), integrin subunit alpha 5 (ITGA5), integrin subunit alpha 6 (ITGA6), integrin subunit alpha 11 (ITGA11), integrin subunit alpha V (ITGAV), integrin subunit beta 2 (ITGB2), integrin subunit beta 5 (ITGB5), integrin subunit beta 8 (ITGB8), integrin alpha 2 beta 1 (ITGA2B1) and integrin alpha 3 beta 1 (ITGA3B1) as the protein moiety and FUCa (1-2) Gal as the glycan moiety. Preferably, the protein moiety is ITGA3, ITGA1, ITGA5, ITGA11, or ITGAV. In an embodiment for quantifying said integrin glycovariant, the binder molecule is Ulex europaeus agglutinin-I (UEA-I) or an antibody specific for the FUCa (1-2) Gal; and the antibody specific for the protein moiety is an anti-ITGA3, an anti-ITGA1, an anti-ITGA2, an anti-ITGA4, an anti-ITGA5, an anti-ITGA6, an anti-ITGA11, an anti-ITGAV, an anti-ITGB2, an anti-ITGB5, an anti-ITGB8, an anti-ITGA2B1, or an anti-ITGA3B1 antibody or an antigen-binding fragment thereof.

Also provided is a kit for use in quantifying a glycosylated antigen of interest having a glycan moiety and a protein moiety. The kit comprises at least a plurality of magnetic particles, each coupled with a plurality of binder molecules specific for said glycan moiety; an antibody or an antigen-binding fragment thereof specific for said protein moiety, labelled with a reactive enzyme; and a substrate capable of creating a detectable signal upon reaction with the reactive enzyme. Optionally, the kit may also comprise a calibrator, i.e. the glycosylated antigen of interest, for preparing a calibration curve. Optionally, the kit may also comprise one or more appropriate controls, an appropriate wash solution and/or an appropriate buffer. Optionally, the kit may also comprise a reaction container, such as a microtiter plate or a reaction chamber of a microfluidistic device. Depending on the selected reactive enzyme or other particulars of the quantification assay in question, the kit may also comprise additional reagents such as stabilizers or agents that enhance or enable the reaction between the reactive enzyme and its substrate, or intensify the signal created. For example, hydrogen peroxide may be included as an oxidizing agent when the selected reactive enzyme is HRP. Further components may also be included, as is readily understood by those skilled in the art.

The more detailed features of each component or element comprised in the kit will become apparent from the above disclosure of the present method, and are thus not repeated here.

The glycosylated target analyte to be quantified by the kit is not particularly limited. Embodiments of the kit include those, in which the target analyte is selected from the target analytes discussed above. Those skilled in the art know how to adjust the components to be included in the kit depending on the target analyte to be quantified.

As used herein, the meaning of a singular noun includes that of a plural noun and thus a singular term, unless otherwise specified, may also carry the meaning of its plural form. In other words, the term “a” or “an” may mean one or more.

As used herein, the terms “one or more” and “at least one” are interchangeable, and can refer to exactly one or to multiple, such as two, three, or even more. The term “a plurality of” refers to two or more.

As used herein, the terms “first”, “second” and the like are merely for the descriptive purpose but cannot be understood as indicating or implying a relative importance.

As used herein, the term “and/or” in a phase such as “X and/or Y” means either “X and Y” or “X or Y” and shall be taken to provide explicit support for both meanings or for either meaning.

The terms “comprising”, “including” and “having” are used herein interchangeably, and are intended to be construed in a non-exclusive manner, i.e. allowing for features not explicitly described also to be present.

### EXPERIMENTAL PART

The following Examples relate to the quantification of CA125-STn, i.e. a glycosylated analyte having STn antigen as the glycan moiety and CA125 as the protein moiety. As a reagent specific for the protein moiety of the analyte, a monoclonal HRP-labeled anti-CA125 antibody was used. As a reagent specific for the glycan moiety of the analyte, a monoclonal anti-STn antigen antibody conjugated to superparamagnetic particles (LodeStars High Bind Carboxyl 2.7 μm) was used. As a signal-producing substrate for HRP, enhanced luminol-based SuperSignal™ ELISA Pico Chemiluminescent Substrate (Thermo Fisher Scientific, USA) was used. Ovarian cancer associated CA125 was used as a calibrator (0, 10, 40, 200 and 500 U/ml. The assays were carried out using LBS OptiPlate-96 (PerkinElmer, USA) 96-well plates. Each measurement was made in three parallel measurements.

### Example 1. Assay with One Separation Cycle (One-Step Assay)

For obtaining a calibration curve for a one-step assay, the calibrators, the magnetic particles and the HRP-labeled anti-CA125 antibody were pipetted into the wells of the microtiter plate so that the final volume in each well was 150 μl, the amount of the magnetic particles was 10 μg, while the amount of the anti-CA125 antibody was 200 ng. The calibrators were with a dilution ratio of 1:10. The plates were then incubated at room temperature with shaking (900 RPM) for 60 min. After incubation, the plate was placed on a magnetic stand and washed with a plate washer. The washing program washed the wells five times with a break for 120 seconds between each wash cycle. After washing, 150 μl of the substrate solution prepared according to the manufacturer's instructions was added into each well, followed by incubation for 1-5 min with shaking (900 RPM). After incubation, luminescence was measured with a plate reader with a measurement time of 0.5-3 seconds. The results were then used for making a calibration curve having the known calibrator concentrations on the x-axis and corresponding chemiluminescence signal intensities on the y-axis.

The one-step assay was tested with normal human serum samples spiked with CA125-STn to a concentration of 0 U/ml or 100 U/ml. First, the spiked serum samples, the HRP-labeled anti-CA125 antibody, and the magnetic particles were added into the wells of a microtiter plate. The sample dilution ratio was 1:10, the amount of the magnetic particles in each well was 10 μg, and the amount of the anti-CA125 antibody was 200 ng. The samples were incubated for 60 min, followed by washing of the wells with a plate washer for five times with 120-second breaks. After washing, 150 μl of the substrate was added into each well, followed by incubation for 5 minutes. Finally, the chemiluminescence was measured with a measurement time of 1 second, and the measure signal intensities were plotted on the calibration curve.

According to the results, both serum samples gave detectable signals. When plotted on the calibration curve, the 0 U/ml sample corresponded to a concentration of 77 U/ml of the calibrator, whereas the 100 U/ml sample corresponded to a concentration of 118 U/ml of the calibrator. The analytical sensitivity of the assay was 0.1 U/ml.

CA125-STn was quantified also in four clinical serum samples using the one-step assay. One of the serum samples was obtained from a subject with ovarian cancer, one was obtained from a subject with benign tumor, one was obtained from a subject with endometriosis, and one was obtained from a healthy subject.

For analyzing the clinical serum samples, magnetic particles and the HRP-labeled anti-CA125 antibody were added to the wells so that the amount of the particles in each well was 10 μg and the amount of the antibody was 200 ng. The clinical samples were then added into the wells so that the total volume was 150 μl and the dilution ratio was 1:10. The plates were incubated for 60 min, followed by washing with a plate washer for five times with 120 second intervals. After washing, 150 μl of the chemiluminescent substrate was added into each well, followed by incubation for 5 minutes. Finally, the luminescence was measured using a plate reader with a measurement time of 1 second, and the concentrations of CA125-STn in the samples were determined by plotting the measured signal intensities on the calibration curve. The results are shown in Table 1 below.

TABLE 1

| Clinical sample | CA15-STn concentration | |
|---|---|---|
| Ovarian cancer | 162.4 | U/ml |
| Bening tumor | 102.6 | U/ml |
| Endometriosis | 5.2 | U/ml |
| Healthy subject | 6.4 | U/ml |

### Example 2. Assay with Two Separation Cycles (Two-Step Assay)

For obtaining a calibration curve for a two-step assay (i.e. in an assay containing two separation cycles), the magnetic particles and the CA125 calibrators were pipetted into the wells of the microtiter plate such that the final volume was 150 μl. The amount of the magnetic particles in each well was 5-20 μg. The calibrators were used with a dilution ratio of 1:10. The plates were then incubated using a plate shaker for 30-60 min at room temperature at a shaking speed of 900 RPM. The shaking speed was so high that the particles did not sink to the bottom of the well, i.e. remained in suspension. The plates were then placed on a 3D-printed magnetic holder and washed with a plate washer. The washing program washed the plates 3-5 times with 250 μl of a washing buffer. There was a 90-120 second break between each was wash cycle, during which break the particles were pulled to the bottom of the wells. Next, 150 μl of an HRP-labeled antibody was applied into the wells so that the amount of the antibody in each well was 25-75 ng. Thereafter, the plates were incubated for 30-60 min with shaking (900 RPM). After the incubation, the plates were placed on a magnetic holder and washed with the washing program disclosed above. After washing, 150 μl of the substrate solution prepared according to the manufacturer's instructions was added into each well, followed by incubation for 1-5 min with shaking (900 RPM). Finally, luminescence below 700 nm was measured for 0.5-3 seconds with a VICTOR® or Nivo™ plate reader (PerkinElmer).

Chromogenic substrates were also tested in the two-step assay format. For this purpose, the chemiluminescent substrate was replaced with a commercially available color-forming 3,3′,5,5′-tetramethylbenzidine (TMB) substrate. Magnetic particles and calibrators were applied into the wells so that the final volume was 150 μl, the amount of the magnetic particles in each well was 10 μg and the dilution ratio of the calibrator was 1:10. The first incubation lasted 30 min with shaking (900 RPM), after which the plates were placed on a magnetic stand and washed four times with 120 second intervals. After washing, 150 μl of the HRP-labeled anti-CA125 antibody diluted was added into each well so that the final amount of the antibody was 50 ng/well. The plates were then incubated for 30 min with shaking (900 RPM), after which the plates were washed using the same washing program as after the first incubation. After washing, 150 μl of the TMB substrate was added into each well, followed incubation in the dark for 10 min with shaking (900 RPM). Thereafter, 50 μl of H₂SO₄ stop solution was added into each well. Next, the reaction solutions were transferred into clear wells, followed by measuring the 450 nm absorbance using a VICTOR® or Nivo™ plate reader.

The two-step assay was also tested with normal human serum samples spiked with the CA125-STn glycovariant to a concentration of 40 U/ml. Magnetic particles and the serum samples were added to the wells of a microtiter plate so that the dilution ratio of the serum samples was 1:10 and the amount of the magnetic particles was 10 μg, the total volume in each well being 150 μl. The first incubation was carried out for 45 min.

After incubation, the plates were washed five times with 90-second intervals. After washing, 150 μl of the HRP-labeled anti-CA125 antibody was added into each well, corresponding to 300 ng of the labeled antibody in each well. The second incubation was carried out for 60 min. After the second incubation, the wells were washed with the same washing program as after the first incubation. Next, 150 μl of the chemiluminescent substrate was added into each well, followed by incubation for 5 min. Finally, the luminescence was measured with a plate reader with a measurement time of 1 second.

According to the results, the 40 U/ml serum sample showed a higher signal than the 10 U/ml calibrator, but a lower signal than the 40 U/ml calibrator. Plotting on the calibration curve indicated that the CA125-STn concentration in the spiked serum sample was 17 U/ml. The analytical sensitivity of the assay was 0.6 U/ml.

1. A method for quantifying at least a first glycosylated analyte having a first glycan moiety and a first protein moiety, in a sample using an immunoassay, the method comprising:
contacting the sample with
a plurality of first magnetic particles, each coupled with a plurality of same or different binder molecules specific for said first glycan moiety, and
a first antibody or an antigen-binding fragment thereof specific for said first protein moiety, labelled with a reactive enzyme;
allowing the first magnetic particles to capture the first glycosylated analyte through specific binding to the first glycan moiety;
allowing the first antibody or the antigen-binding fragment thereof to form a first immunocomplex with the captured first glycosylated analyte through specific binding to the first protein moiety;
separating captured analytes from any uncaptured analytes and unbound antibodies or antigen-binding fragments thereof using an external magnetic field, optionally combined with one or more wash cycles;
adding a first substrate and allowing it to react with the reactive enzyme of the labelled first antibody or the antigen-binding fragment thereof in the first immunocomplex, thereby resulting in a first detectable signal;
measuring the intensity of the first detectable signal; and
correlating the measured intensity of the first detectable signal to the quantity of the first glycosylated analyte in the sample;
or
contacting the sample with
a plurality of first magnetic particles, each coupled with a plurality of same or different binder molecules specific for said first glycan moiety;
allowing the first magnetic particles to capture the first glycosylated analyte through specific binding to the first glycan moiety;
separating the captured analyte from any uncaptured analytes using an external magnetic field, optionally combined with one or more wash cycles;
contacting the captured analyte with
a first antibody or an antigen-binding fragment thereof specific for said first protein moiety, labelled with a reactive enzyme;
allowing the first antibody or the antigen-binding fragment thereof to form a first immunocomplex with the captured analyte through specific binding to the first protein moiety;
removing any unbound antibodies or antigen-binding fragments thereof using an external magnetic field, optionally with one or more wash cycles;
adding a first substrate and allowing it to react with the reactive enzyme of the first labelled antibody or the antigen-binding fragment thereof in the immunocomplex, thereby resulting in a first detectable signal;
measuring the intensity of the first detectable signal; and
correlating the measured intensity of the first detectable signal to the quantity of the first glycosylated analyte in the sample.

2. The method according to claim 1, wherein the correlating step is accomplished by plotting a calibration curve based on known first glycosylated analyte concentrations on the x-axis and corresponding signal intensities on the y-axis, wherein the quantity of the first glycosylated analyte in the sample is determined using the calibration curve and the measured signal intensity.

3. The method according to claim 1, further comprising quantifying at least a second glycosylated analyte having a second glycan moiety and a second protein moiety, in the sample, the method comprising:
contacting the sample with
a plurality of second magnetic particles, each coupled with a plurality of same or different binder molecules specific for said second glycan moiety, and
a second antibody or an antigen-binding fragment thereof specific for said second protein moiety, labelled with a reactive enzyme that is different from the reactive enzyme of the first antibody or the antigen-binding fragment thereof;
allowing the second magnetic particles to capture the second glycosylated analyte through specific binding to the second glycan moiety;
allowing the second antibody to form a second immunocomplex with the captured second glycosylated analyte through specific binding to the second protein moiety;
separating captured analytes from any uncaptured analytes and unbound antibodies or the antigen-binding fragments thereof using an external magnetic field, optionally combined with one or more wash cycles;
adding a second substrate that is different from the first substrate and allowing it to react with the reactive enzyme of the labelled second antibody or antigen-binding fragment thereof in the second immunocomplex, thereby resulting in a second detectable signal that is distinguishable from the first signal;
measuring the intensity of the second detectable signal; and
correlating the measured intensity of the second detectable signal to the quantity of the second glycosylated analyte in the sample;
or
contacting the sample with
a plurality of second magnetic particles, each coupled with a plurality of same or different binder molecules specific for said second glycan moiety;
allowing the second magnetic particles to capture the second glycosylated analyte through specific binding to the second glycan moiety;
separating the captured analyte from any uncaptured analytes using an external magnetic field, optionally combined with one or more wash cycles;
contacting the captured analyte with
a second antibody or an antigen-binding fragment thereof specific for said second protein moiety, labelled with a reactive enzyme that is different from the reactive enzyme of the first antibody or the antigen-binding fragment thereof;
allowing the second antibody or the antigen-binding fragment thereof to form a second immunocomplex with the captured analyte through specific binding to the second protein moiety;
removing any unbound antibodies or antigen-binding fragments thereof using an external magnetic field, optionally with one or more wash cycles;
adding a second substrate that is different from the first substrate and allowing it to react with the reactive enzyme of the second labelled antibody or the antigen-binding fragment thereof in the second immunocomplex, thereby resulting in a second detectable signal that is distinguishable from the first signal;
measuring the intensity of the second detectable signal; and
correlating the measured intensity of the second detectable signal to the quantity of the second glycosylated analyte in the sample.

4. The method according to claim 3, wherein the correlating step is accomplished by plotting a calibration curve based on known second glycosylated analyte concentrations on the x-axis and corresponding signal intensities on the y-axis, wherein the quantity of the second glycosylated analyte in the sample is determined using the calibration curve and the measured signal intensity.

5. The method according to claim 3, wherein the quantifying of the first and the second glycosylated analytes is carried out by multiplexing.

6. The method according to claim 1, wherein the binder molecule is lectin or a glycan-specific antibody.

7. The method according to claim 1, wherein the reactive enzyme is alkaline phosphatase (AP) or horseradish peroxidase (HRP).\

8. The method according to claim 1, wherein the glycosylated analyte is CA125-STn having a sialyl Tn antigen (STn) as the glycan moiety and cancer antigen 125 (CA125) as the protein moiety.

9. The method according to claim 8, wherein the binder molecule is an anti-STn antibody or macrophage galactose-type lectin (MGL), and wherein the antibody specific for the protein moiety is an anti-CA125 antibody or an antigen-binding fragment thereof.

10. The method according to claim 1, wherein the glycosylated analyte is CA125-Tn having a Tn antigen (Tn) as the glycan moiety CA125 as the protein moiety.

11. The method according to claim 10, wherein the binder molecule is an anti-Tn antibody or MGL, and wherein the antibody specific for the protein moiety is an anti-CA125 antibody or an antigen-binding fragment thereof.

12. The method according to claim 1, wherein the glycosylated target analyte is a CEA glycovariant having CEA as the protein moiety and a glycan which is capable of specific binding to mannose binding lectin (MBL), Dendritic Cell-Specific Intercellular adhesion molecule-3-Grabbing Non-integrin (DC-SIGN) or to MGL as the glycan moiety.

13. The method according to claim 12, wherein the binder molecule is MBL, DC-SIGN, MBL, or an antibody capable to specific binding to the same glycan moiety as MBL, DC-SIGN or MBL; and wherein the antibody specific for the protein moiety is an anti-CEA antibody or an antigen-binding fragment thereof.

14. The method according to claim 1, wherein the glycosylated target analyte is a CA15-3 glycovariant having CA15-3 as the protein moiety and a glycan which is capable of specific binding to MGL or wheat germ agglutin (WGA) as the glycan moiety.

15. The method according to claim 14, wherein the binder molecule is MGL, WGA, or an antibody capable to specific binding to the same glycan moiety as MGL or WGA; and wherein the antibody specific for the protein moiety is an anti-CA15-3 antibody or an antigen-binding fragment thereof.

16. The method according to claim 1, wherein the glycosylated target analyte is a prostate-specific antigen (PSA) glycovariant having PSA as the protein moiety and a glycan which is capable of specific binding to MGL as the glycan moiety.

17. The method according to claim 16, wherein the binder molecule is MGL, or an antibody capable to specific binding to the same glycan moiety as MGL; and wherein the antibody specific for the protein moiety is an anti-PSA antibody or an antigen-binding fragment thereof.

18. The method according to claim 1, wherein the glycosylated target analyte is an integrin glycovariant having integrin selected from integrin subunit alpha 3 (ITGA3), integrin subunit alpha 1 (ITGA1), integrin subunit alpha 2 (ITGA2), integrin subunit alpha 4 (ITGA4), integrin subunit alpha 5 (ITGA5), integrin subunit alpha 6 (ITGA6), integrin subunit alpha 11 (ITGA11), integrin subunit alpha V (ITGAV), integrin subunit beta 2 (ITGB2), integrin subunit beta 5 (ITGB5), integrin subunit beta 8 (ITGB8), integrin alpha 2 beta 1 (ITGA2B1) and integrin alpha 3 beta 1 (ITGA3B1) as the protein moiety and FUCa (1-2) Gal as the glycan moiety.

19. The method according to claim 18, wherein the binder molecule is Ulex europaeus agglutinin-I (UEA-I) or an antibody specific for the FUCa (1-2) Gal; and wherein the antibody specific for the protein moiety is an anti-ITGA3, an anti-ITGA1, an anti-ITGA2, an anti-ITGA4, an anti-ITGA5, an anti-ITGA6, an anti-ITGA11, an anti-ITGAV, an anti-ITGB2, an anti-ITGB5, an anti-ITGB8, an anti-ITGA2B1, or an anti-ITGA3B1 antibody or an antigen-binding fragment thereof.

20. An immunoassay kit for quantifying a glycosylated analyte having a glycan moiety and a protein moiety, in a sample, comprising:
a plurality of magnetic particles, each coupled with a plurality of binder molecules specific for said glycan moiety;
an antibody or an antigen-binding fragment thereof specific for said protein moiety, labelled with a reactive enzyme; and
a substrate capable of creating a detectable signal upon reaction with the reactive enzyme.
